(19) United States
(12) Patent Application Publication
(10) Pub. No.: US 2024/0032828 A1
(43) Pub. Date: Feb. 1, 2024

(54) BIOMETRIC INFORMATION DETECTION METHOD AND ELECTRONIC DEVICE FOR IMPLEMENTING SAME

(71) Applicant: SAMSUNG ELECTRONICS CO., LTD., Suwon-si, Gyeonggi-do (KR)

(72) Inventors: Hyunjun JUNG, Suwon-si, Gyeonggi-do (KR); Hyejung SEO, Suwon-si, Gyeonggi-do (KR); Suho LEE, Suwon-si, Gyeonggi-do (KR); Seongmin JE, Suwon-si, Gyeonggi-do (KR)

(21) Appl. No.: 18/380,061

(22) Filed: Oct. 13, 2023

Related U.S. Application Data
(63) Continuation of application No. PCT/KR2022/002088, filed on Feb. 11, 2022.

(30) Foreign Application Priority Data
Apr. 13, 2021 (KR) 10-2021-0047660

(51) Int. Cl. A61B 5/1455; A61B 5/026; A61B 5/0205

(52) U.S. Cl. CPC A61B 5/14551; A61B 5/0261; A61B 5/0205; A61B 5/02416

(57) ABSTRACT

An electronic device includes an output device, a biometric sensor including a light-emitting unit for emitting light at a first portion of a subject, and a light-receiving unit for receiving light emitted by the light-emitting unit and a processor operatively connected to the output device and the biometric sensor, wherein the processor can be configured to acquire a perfusion index on the basis of a photoplethysmogram (PPG) signal acquired through the biometric sensor, measure oxygen saturation by using the PPG signal if the perfusion index corresponds to a first criterion, and output, through the output device, guide information instructing an occlusion operation for a second portion, differing from the first portion of the subject, if the perfusion index corresponds to a second criterion.

### CROSS-REFERENCE TO RELATED APPLICATIONS

This application is a continuation application, claiming priority under § 365(c), of International Application No. PCT/KR2022/002088, filed on Feb. 11, 2022, which is based on and claims the benefit of Korean patent application number 10-2021-0047660, filed on Apr. 13, 2021, in the Korean Intellectual Property Office, the disclosures of which are incorporated by reference herein in their entireties.

### TECHNICAL FIELD

This disclosure relates to an electronic device, and more particularly, to a biometric information detection method and an electronic device implementing the same.

### BACKGROUND ART

While performing the same or more diverse functions, electronic devices have been gradually miniaturized and developed to be easily portable. Although such electronic devices are generally kept in a user's pocket and carried, the electronic devices may be worn on a wrist or worn on a head portion or arm of a human body.

In addition, electronic devices may be equipped with biometric sensors (e.g., health care sensors) to measure biometric information (e.g., health data). For example, an electronic device may measure oxygen saturation by analyzing a photoplethysmogram (PPG) signal obtained through a PPG sensor.

### DISCLOSURE

### Technical Problem

The aforementioned electronic device may measure oxygen saturation based on a component ratio of infrared (IR) light and red light of the PPG signal detected through a PPG sensor. However, measurement accuracy of oxygen saturation may vary depending on a perfusion index (PI) calculated by a magnitude ratio of an AC component to a DC component of a PPG signal. For example, the oxygen saturation measured under a relatively low perfusion index may have lower accuracy than oxygen saturation measured under a high perfusion index.

Accordingly, when the perfusion index is low, the electronic device may improve measurement accuracy by measuring oxygen saturation based on a change in the DC component of the PPG signal.

However, in order to identify the variation of the DC component, a change in blood flow must be induced for a long time, and even a small movement of the subject may act as noise.

Therefore, one or more various embodiments provide a biometric information detection method capable of inducing an occlusion operation such that a change in a DC component of a PPG signal increases when a perfusion index is low, and an electronic device for implementing the same.

### Technical Solution

According to various embodiments, an electronic device includes an output device, a biometric sensor including a light emitting unit that emits light to a first part of a subject and a light receiving unit that receives light emitted by the light emitting unit, and a processor operably connected with the output device and the biometric sensor, wherein the processor may be configured to obtain a perfusion index based on a photoplethysmogram (PPG) signal obtained through the biometric sensor, measure oxygen saturation by using the PPG signal when the perfusion index corresponds to a first reference, and output guide information instructing an occlusion operation for a second part different from the first part of the subject through the output device when the perfusion index corresponds to a second reference lower than the first reference.

According to various embodiments, a method of operating an electronic device includes obtaining a photoplethysmogram (PPG) signal of a first part of a subject through a biometric sensor of the electronic device, obtaining a perfusion index based on the PPG signal, measuring oxygen saturation by using the PPG signal when the perfusion index corresponds to a first reference, and outputting guide information instructing an occlusion operation for a second part different from the first part of the subject through the output device when the perfusion index corresponds to a second reference lower than the first reference.

According to various embodiments, a biometric information measurement system includes an electronic device including a biometric sensor configured to obtain a biometric signal by contacting a first part of a body, and an external device including a fastening member configured to be coupled to a second part different from the first part of the body and a driving module configured to control movement of the fastening member, wherein the electronic device may be configured to measure oxygen saturation by using the biometric signal when a perfusion index of the biometric signal corresponds to a first reference, and instruct an occlusion operation to the external device when the perfusion index corresponds to a second reference lower than the first reference, and wherein the external device may be configured to move the fastening member to compress the second part of the body through the driving module in response to an instruction of the electronic device.

### Advantageous Effects

According to various embodiments, when the perfusion index is low, the electronic device may improve oxygen saturation measurement performance by inducing an occlusion operation that reduces blood flow and increasing a change in the DC component of a PPG signal.

Effects obtained by various embodiments of the invention may not be limited to the above.

### DESCRIPTION OF DRAWINGS

FIG. 1 is a block diagram illustrating an electronic device in a network environment, according to various embodiments;

FIG. 2A is a diagram illustrating a configuration of an electronic device, according to various embodiments;

FIG. 2B is a diagram illustrating the shape of a PPG signal, according to various embodiments;

FIG. 2C is a diagram illustrating the shape of a PPG signal, according to various embodiments;

FIG. 2D is a diagram illustrating the shape of a PPG signal, according to various embodiments;

FIG. 3A is a diagram illustrating an electronic device, according to various embodiments;

FIG. 3B is a diagram illustrating information related to a biometric information obtaining operation provided through an electronic device, according to various embodiments;

FIG. 4 is a flowchart illustrating an operation of measuring biometric information of an electronic device, according to various embodiments;

FIG. 5 is a flowchart illustrating an operation of controlling an occlusion operation in an electronic device, according to various embodiments;

FIG. 6 is a flowchart illustrating another operation of controlling an occlusion operation in an electronic device, according to various embodiments;

FIG. 7 is a diagram illustrating a configuration of a biometric information measurement system, according to various embodiments;

FIG. 8A is a diagram illustrating an occlusion device, according to various embodiments;

FIG. 8B is a diagram illustrating information related to a biometric information obtaining operation provided through an occlusion device, according to various embodiments;

FIG. 9 is a flowchart illustrating another operation of measuring biometric information in an electronic device, according to various embodiments;

FIG. 10 is a flowchart illustrating an operation of controlling an occlusion operation in an electronic device, according to various embodiments;

FIG. 11 is a block diagram illustrating another configuration of a biometric information measurement system, according to various embodiments; and

FIG. 12 is a diagram illustrating a method of measuring biometric information of a biometric information measurement system according to various embodiments.

With regards to the description of drawings, the same or similar elements may be marked by the same or similar reference numerals.

### MODE FOR INVENTION

Various embodiments of the invention may be described with reference to accompanying drawings. Accordingly, those of ordinary skill in the art will recognize that modification, equivalent, and/or alternative of the various embodiments described herein can be variously made without departing from the scope and spirit of the invention. With regard to description of drawings, similar elements may be marked by similar reference numerals.

It will be understood that when an element is referred to as being related to another element such as being “on” another element, it can be directly on the other element or intervening elements may be present therebetween. In contrast, when an element is referred to as being related to another element such as being “directly on” another element, there are no intervening elements present.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “At least one” is not to be construed as limiting “a” or “an.” “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

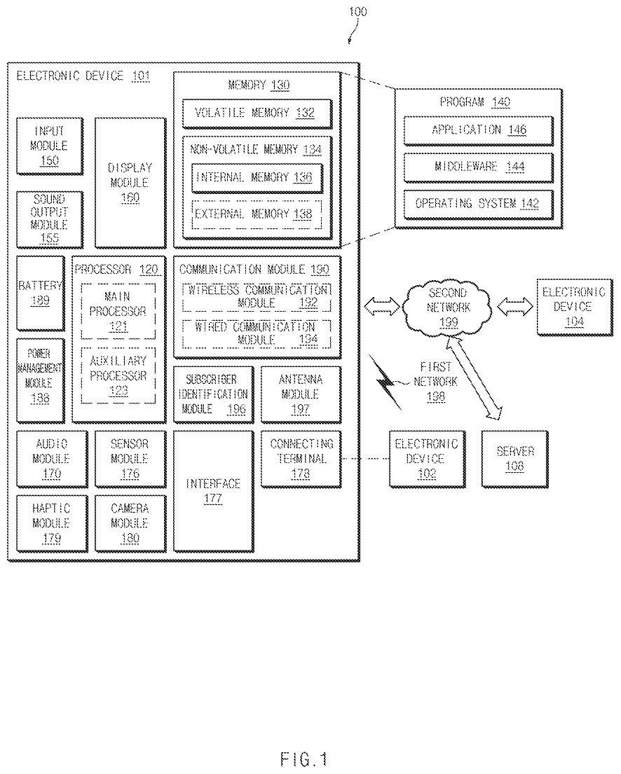

FIG. 1 is a block diagram illustrating an electronic device 101 in a network environment 100 according to various embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™ wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

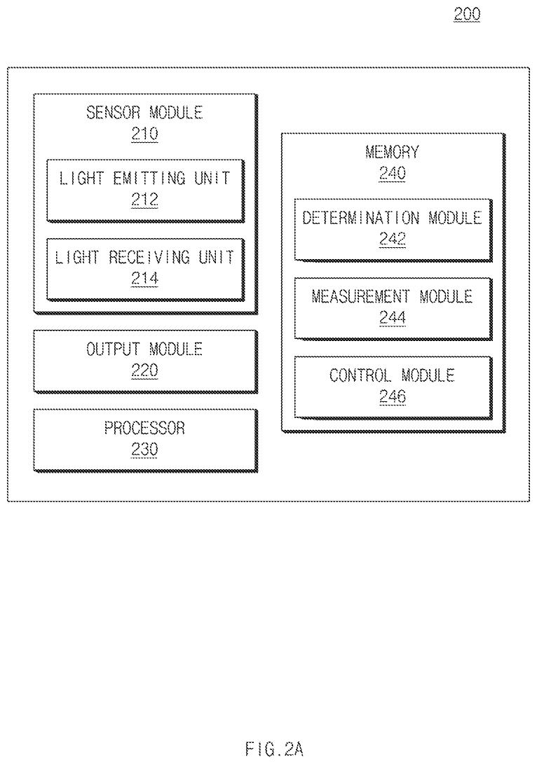

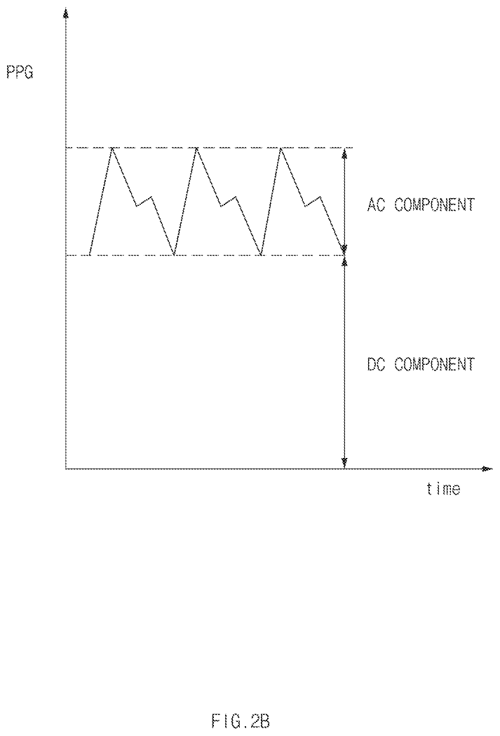

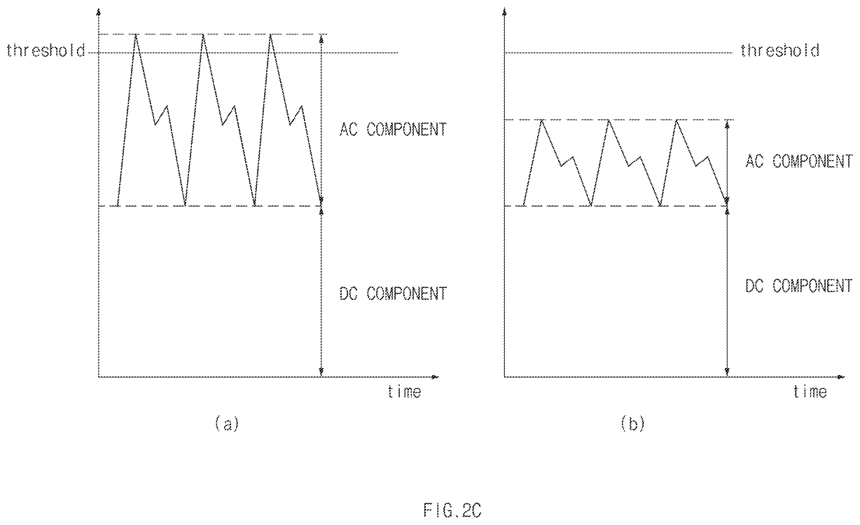

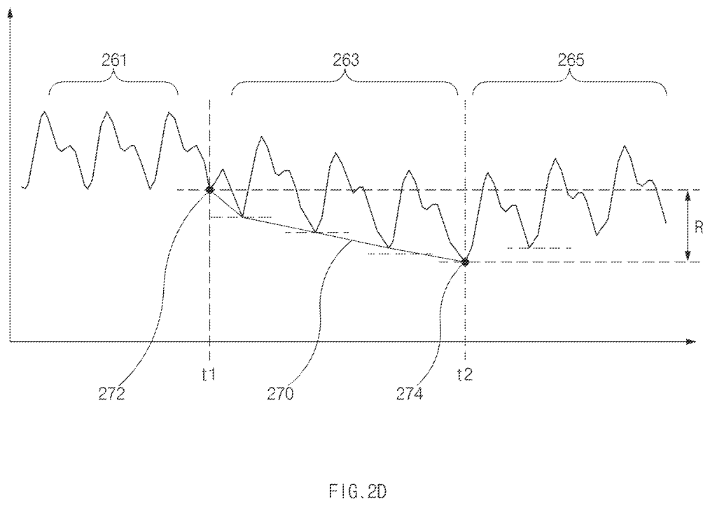

FIG. 2A is a diagram illustrating a configuration of an electronic device, according to various embodiments. FIGS. 2B to 2D are diagrams illustrating the shape of a PPG signal, according to various embodiments.

Referring to FIG. 2A, an electronic device 200 according to various embodiments may include a sensor module 210, an output module 220, a processor 230, and a memory 240.

The components of the above-described electronic device 200 are proposed as an example, and embodiments are not limited thereto. For example, in an embodiment, the electronic device 200 may be implemented with more or fewer components than those illustrated in FIG. 2A. For example, the electronic device 200 may be the electronic device 101 shown in FIG. 1, and may include at least one input module (e.g., the input module 150), at least one camera module (e.g., the camera module 180), a power management module (e.g., the power management module 188), the communication module 190, a housing that forms the appearance of the electronic device 200 and protects the above-described components from external shock, and the like as components of the electronic device 200.

According to various embodiments, the sensor module 210 may measure a pulse wave signal of a subject (e.g., a body part such as a wrist or a finger). The pulse wave signal may include a photoplethysmogram (PPG) signal. For example, the sensor module 210 may include a light emitting unit 212 and a light receiving unit 214.

According to an embodiment, the light emitting unit 212 may include a first light emitting device for emitting light of a first wavelength (e.g., red (wavelength: about 600 nm to about 700 nm)) to a subject and a second light emitting device for emitting light of a second wavelength (e.g., infrared light (wavelength: about 780 nm to about 1000 μm)) to the subject. For example, the first light emitting device and the second light emitting device constituting the light emitting unit 212 may be implemented using a light emitting diode (LED), an organic light emitting diode (OLED), a quantum dot light emitting diode (QLED), a laser diode, and/or phosphors. However, this is only illustrative, and embodiments are not limited thereto. For example, the light emitting unit 212 may further include one or more additional light emitting devices for emitting light of the same or different wavelengths (e.g., blue wavelength, green wavelength, and the like) from those of the first light emitting device and the second light emitting device.

According to an embodiment, the light receiving unit 214 may receive light and generate a current signal by photoelectric conversion of the received light. For example, the light receiving unit 214 may include at least one light receiving device for detecting light of the first wavelength emitted from the first light emitting device and light of the second wavelength emitted from the second light emitting device. For example, at least one light receiving device may include a photo detector or a photo diode.

For example, in an embodiment, the light receiving unit 214 may detect light emitted from the light emitting unit 212 (e.g., the first light emitting device and the second light emitting device) and reflected from the subject. In this regard, the light receiving unit 214 and the light emitting unit 212 may be arranged on the same surface.

As another example, in an embodiment, the light receiving unit 214 may detect light emitted from the light emitting unit 212 and transmitted through the subject. In this regard, the light receiving unit 214 and the light emitting unit 212 may be arranged to face each other. However, this is only illustrative, and embodiments are not limited thereto, and the light receiving unit 214 and the light emitting unit 212 may be arranged in various forms.

According to various embodiments, the output module 220 may output various information based on control of the processor 230. At least some pieces of the various types of information may be related to a biometric information acquisition operation. For example, the output module 220, which is an output device that outputs auditory information, tactile information, and/or visual information, may include an audio output module (e.g., the audio output module 155), a haptic module (e.g., the haptic module 179) and/or a display module (e.g., the display module 160).

According to various embodiments, the processor 230 may be operatively connected to the sensor module 210, the output module 220, and the memory 240, and various components (e.g., hardware or software components) of the electronic device 200.

According to an embodiment, the processor 230 may obtain biometric information based on information (e.g., a PPG signal) obtained through the sensor module 210. The biometric information may include oxygen saturation. However, this is only illustrative, and embodiments are not limited thereto. For example, the processor 230 may measure various types of biometric information such as blood pressure, heart rate, electrocardiogram, and/or skin moisture by using the PPG signal.

According to an embodiment, the processor 230 may measure biometric information by extracting an AC component (or AC signal) and a DC component (or DC signal) from the PPG signal sensed through the light receiving unit 214. As shown in FIG. 2B, in an embodiment, the PPG signal may be divided into an AC component representing the absorption of pulsatile arterial blood due to heartbeat and a DC component due to absorption of non-pulsating arterial blood, venous blood, skin, bone, tissue, and the like. Because a process of extracting an AC component and a DC component from a PPG signal is well known in the art, the details will be omitted.

For example, in an embodiment, the PPG signal may include a first PPG signal related to light of a first wavelength (e.g., red) and a second PPG signal related to light of a second wavelength (e.g., infrared rays). As an example of biometric information, in an embodiment, the processor 230 may measure oxygen saturation (SpO2) based on the following Equation 1:

$$\begin{matrix}{Sp O}_{2} = 110 - 25 \times {r}_{i} , \\ {r}_{i} = \frac{{AC}_{RED} / {DC}_{RED}}{{AC}_{IR} / {DC}_{IR}}\end{matrix}$$

where AC_{RED} represents the AC component of the first PPG signal measured by the first wavelength (e.g., red), DC_{RED} represents the DC component of the first PPG signal, AC IR represents the AC component of the second PPG signal measured by a second wavelength (e.g., infrared rays), and DC_{IR} represents a DC component of the second PPG signal.

According to an embodiment, the processor 230 may determine the effectiveness of the AC component in measuring oxygen saturation. The effectiveness determination may include determining whether oxygen saturation can be measured with a certain level of accuracy for the AC component.

For example, as shown in (a) of FIG. 2C, the processor 230 may determine that an AC component having an amplitude greater than a specified threshold is an AC component effective for measuring oxygen saturation. To the contrary, as shown in (b) of FIG. 2C, the processor 230 may determine that an AC component having an amplitude smaller than a specified magnitude is an AC component that is not effective in measuring oxygen saturation.

In this regard, in an embodiment, the processor 230 may determine the effectiveness of the AC component based on a perfusion index (PI). A case in which the amplitude of the AC component is less than a specified threshold may include a situation in which a perfusion index less than a threshold index (e.g., approximately about 0.5%) is obtained. To the contrary, a case in which the amplitude of the AC component is greater than a specified threshold may include a situation in which a perfusion index equal to or greater than the threshold index is obtained.

For example, in an embodiment, the processor 230 may perform an operation of checking a perfusion index and comparing the perfusion index with a specified threshold index, based on the ratio of the AC signal value of the first PPG signal related to the light of the first wavelength (e.g., red) to the magnitude of the DC signal value (e.g., AC signal value of the first PPG/DC signal value of the first PPG*100) and the ratio of the AC signal value of the second PPG signal related to the light of the second wavelength (e.g., infrared) to the magnitude of the DC signal value (e.g., AC signal value of the second PPG/DC signal value of the second PPG*100). However, this is only illustrative, and embodiments are not limited thereto. For example, in an embodiment, the processor 230 may perform the effectiveness determination operation by using the perfusion index for the first PPG signal and/or may perform the effectiveness determination operation by using the perfusion index for the second PPG signal.

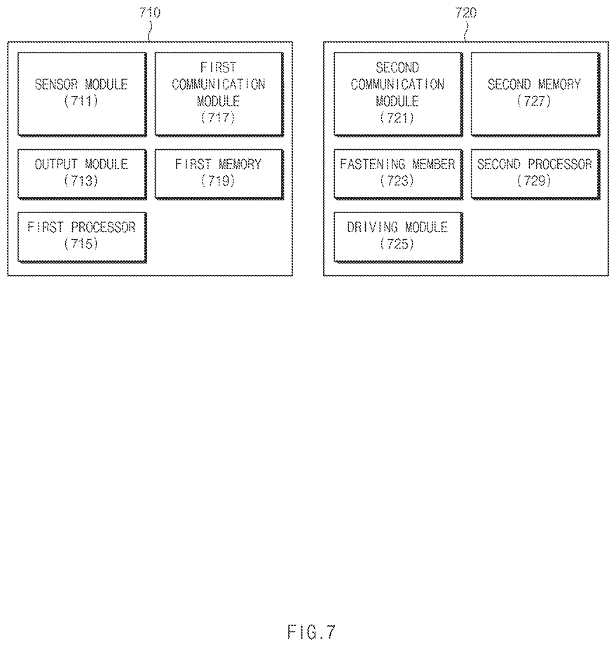

According to an embodiment, when it is determined that the AC component is ineffective, the processor 230 may induce an occlusion operation. The occlusion operation may be an action of compressing a part of the body. For example, in an embodiment, the processor 230 may output at least one piece of visual guide information, auditory guide information and/or tactile guide information and indicating an occlusion operation. Accordingly, the user may press a part (e.g. blood vessel) of the body near the measurement site with the and/hand or perform an occlusion operation of pressing the part of the body by using a separate occlusion device such as a blood pressure cuff. As another example, in an embodiment, the processor 230 may provide information instructing an occlusion operation to at least one external device. For example, as described below with reference to FIG. 7, the processor 230 may instruct an occlusion operation to a wearable external device configured to provide an occlusion function. Accordingly, at least one external device may perform an occlusion operation according to an instruction of the processor 230.

According to an embodiment, after inducing the occlusion operation, the processor 230 may induce occlusion stop based on an occluded PPG signal. For example, the processor 230 may induce occlusion stop in response to detecting a variation amount of a DC component exceeding a reference range (e.g., R in FIG. 2D).

According to an embodiment, after inducing occlusion stop, the processor 230 may measure oxygen saturation based on the occluded PPG signal. The occluded PPG signal may be a PPG signal obtained through the sensor module 210 during or after the occlusion operation is performed. As shown in FIG. 2D, compared with a PPG signal 261 before the occlusion operation is performed, an occluded PPG signal 263 may include a DC component whose variation increases in inverse proportion to the blood flow in the body, which decreases from time point t1 at which the occlusion operation starts. In addition, when the occlusion operation is stopped at t2, the variation amount of the DC component of a PPG signal 265 may decrease due to an increase in blood flow in the body.

For example, in an embodiment, the processor 230 may measure biometric information by obtaining a variation amount of a DC component from the occluded PPG signal. The processor 230 may determine a baseline of the DC component through the occluded PPG signal 263. As shown in FIG. 2D, a baseline 270 may be represented by a line connecting the lowest point of each cycle of the AC component. For example, the processor 230 may measure oxygen saturation based on the ratio of the difference between a baseline 272 (or DC component value) at the time when the occlusion operation starts and a baseline 274 at the time when the occlusion operation ends.

According to various embodiments, the memory 240 may store commands and/or data related to at least one other component of the electronic device 200. According to an embodiment, the memory 240 may include programs, algorithms, routines, and/or instructions related to the biometric information measurement. The memory 240 may include at least one program module instructing each operation of the processor 230 mentioned in the disclosure. The program module may include the program 140 of FIG. 1. According to an embodiment, at least one program module may include a determination module 242, a measurement module 244, and a control module 246. However, this is only illustrative, and embodiments are not limited thereto. For example, in an embodiment, at least one of the aforementioned modules may be excluded from the configuration of the memory 240, and to the contrary, another module other than the aforementioned modules may be added as a component of the memory 240. In addition, some of the above-mentioned modules may be integrated with other modules.

According to an embodiment, the determination module 242 may include an instruction for determining the effectiveness of the AC component in measuring oxygen saturation. According to an embodiment, the measurement module 244 may include an instruction for measuring oxygen saturation based on information (e.g., the PPG signal) obtained through the sensor module 210. According to an embodiment, the control module 246 may include an instruction for controlling output of guide information instructing an occlusion operation.

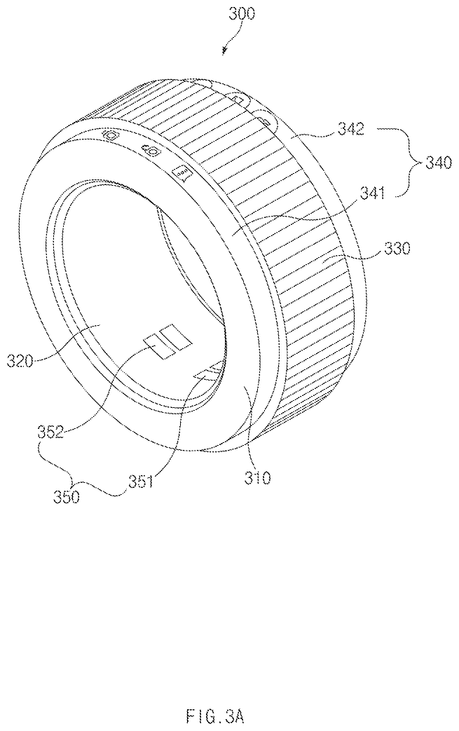

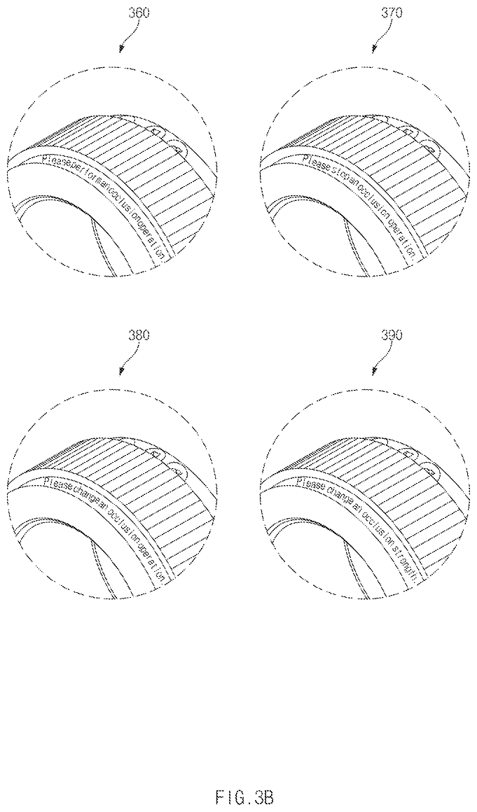

FIG. 3A is a diagram illustrating an electronic device, according to various embodiments. FIG. 3B is a diagram illustrating information related to a biometric information obtaining operation provided through an electronic device, according to various embodiments.

Referring to FIG. 3A, in an embodiment, an electronic device 300 (e.g., the electronic device 200) may be a ring-shaped wearable device worn on the body (e.g., a finger). However, this is only illustrative, and embodiments are not limited thereto. For example, in an embodiment, the electronic device 300 may be implemented as a wearable device in the form of a watch or band.

According to various embodiments, the electronic device 300 may include an outer ring member 310, an inner ring member 320 arranged along an inner circumferential surface of the outer ring member 310, a cover member 330 arranged along an outer circumferential surface of the outer ring member 310, a display 340, and a sensor module 350. However, this is only illustrative, and embodiments are not limited thereto.

According to various embodiments, the outer ring member 310 may have an inner diameter to be fitted to the user's body. In this case, the inner ring member 320 may directly contact the user's body.

According to an embodiment, at least some of the components (e.g., at least one of the output module 220, the processor 230 or the memory 240) described above with reference to FIG. 2A may be arranged in the outer ring member 310. For example, in an embodiment, a groove in which at least some components may be placed may be formed on the outer circumferential surface of the outer ring member 310. For example, at least some components placed on the outer circumferential surface of the outer ring member 310 may not be exposed to the outside by the cover member 330. For example, the outer ring member 310, the inner ring member 320, and/or the cover member 330 may form the appearance of the electronic device 300.

According to various embodiments, the inner ring member 320 may be formed in a circular shape having a specified thickness, and may be detachably coupled along the inner circumferential surface of the outer ring member 310.

According to an embodiment, at least a portion of the sensor module 350 may be exposed through the inner ring member 320. For example, the inner ring member 320 may include a plurality of openings formed to expose at least a portion of a light emitting unit 351 and a light receiving unit 352 of the sensor module 350. Accordingly, in a state where the electronic device 300 is worn on the body, the sensor module 350 may be exposed toward the body through the inner ring member 320.

According to various embodiments, the display 340 may include at least one of a first display 341 and a second display 342 arranged in the circumferential direction of the outer ring member 310 along the outer side of the outer ring member 310 with the cover member 330 interposed therebetween.

According to an embodiment, the display 340 may be configured to provide visual information (e.g., text, image, video, icon, and/or symbol, and the like) to a user and/or receive a user input (e.g., touch input). For example, in an embodiment and as shown in FIG. 3B, the display 340 may output at least one of visual guide information 360 indicating that an occlusion operation is required, visual guide information 370 indicating that occlusion interruption is required, visual guide information 380 indicating that an occlusion position needs to be changed, and/or visual guide information 390 indicating that the occlusion strength needs to be changed. For example, visual guide information such as an icon, image, text, and/or the like indicating an occlusion operation, occlusion stop, occlusion strength change, and/or occlusion position change may be output through the display 340. However, this is only illustrative, and embodiments are not limited thereto. For example, as described above, in an embodiment, the electronic device 300 may additionally or selectively include an audio output module (e.g., the audio output module 155) outputting auditory guide information and/or a haptic module (e.g., the haptic module 179) outputting tactile guide information. In this regard, the electronic device 300 may output occlusion operation guidance, occlusion stop guidance, occlusion position change guidance, and/or occlusion strength change guidance by using different sound patterns and/or vibration patterns. In addition, when outputting occlusion operation guidance, occlusion stop guidance, occlusion position change guidance, and/or occlusion strength change guidance, one output module may be used and/or at least two output modules are used when the electronic device 300 has a plurality of output modules.

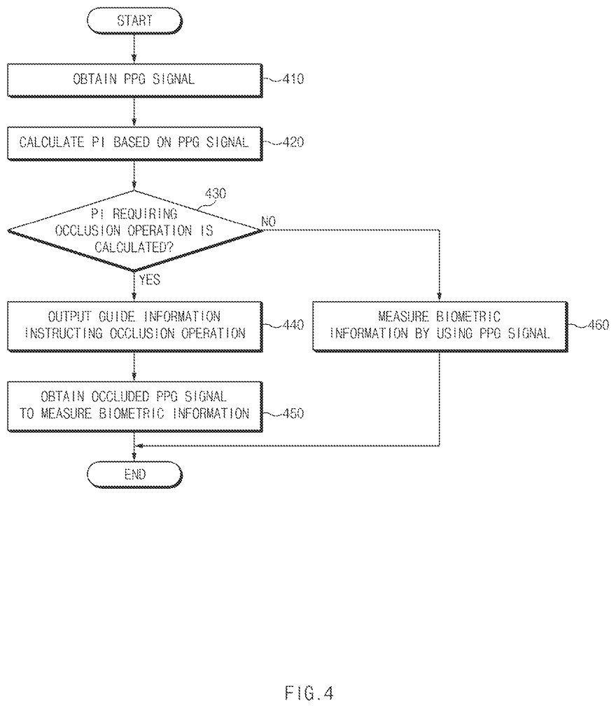

FIG. 4 is a flowchart illustrating an operation of measuring biometric information of an electronic device according to various embodiments. Each operation in the following embodiment may be performed sequentially, but is not necessarily performed sequentially. For example, the order of each operation may be changed, or at least two operations may be performed in parallel. In addition, at least one of the above-described operations may be omitted according to an embodiment.

Referring to FIG. 4, in operation 410, the electronic device 200 (and/or the processor 230) according to various embodiments may obtain a PPG signal. According to an embodiment, the electronic device 200 may obtain a PPG signal while being worn on a part of the user's body. In this regard, the electronic device 200 may emit the light of the first wavelength (e.g., red (wavelength: about 600 nm to about 700 nm)) and the second wavelength (e.g., infrared (wavelength: about 780 nm to about 1000 μm)), and may detect the light of the first wavelength and/or the light of the second wavelength reflected from and/or transmitted through the body.

According to various embodiments, in operation 420, the electronic device 200 (and/or the processor 230) may calculate a perfusion index (PI) based on the PPG signal. According to an embodiment, the electronic device 200 may calculate the perfusion index based on the ratio (e.g., the AC signal value of the first PPG/the DC signal value of the first PPG*100) of the magnitude of the DC signal value to the AC signal value of the first PPG signal related to the light of the first wavelength (e.g., red) and the ratio (e.g., AC signal value of the second PPG/DC signal value of the second PPG*100) of the magnitude of the DC signal value to the AC signal value of the second PPG signal related to the light of the second wavelength (e.g. infrared).

According to various embodiments, in operation 430, the electronic device 200 (and/or the processor 230) may determine whether a PI requiring an occlusion operation is calculated. The occlusion action may be an action of compressing a part of the body. According to an embodiment, the electronic device 200 may determine whether the PI requiring an occlusion operation is calculated by comparing the calculated PI with a specified threshold indicator. The PI requiring an occlusion operation may be calculated in a situation in which an AC component that is not effective for measuring oxygen saturation is included in the PPG signal. For example, in an embodiment, the electronic device 200 may determine that an occlusion operation is required when a PI less than the threshold index is calculated. In addition, in an embodiment, the electronic device 200 may determine that the occlusion operation is unnecessary when a PI equal to or greater than the threshold indicator is calculated.

According to various embodiments, when a PI that does not require an occlusion operation is calculated, in operation 460, the electronic device 200 (and/or the processor 230) may measure biometric information by using the PPG signal. According to an embodiment, the electronic device 200 may measure oxygen saturation based on a component ratio of infrared light and red light extracted from the PPG signal.

According to various embodiments, when a PI requesting the occlusion operation is calculated, in operation 440, the electronic device 200 (and/or the processor 230) may output guide information (e.g., reference number 360 of FIG. 3B) instructing the occlusion operation. For example, in an embodiment, the electronic device 200 may output at least one of visual guide information, auditory guide information, and tactile guide information indicating an occlusion operation. Accordingly, the user may perform an occlusion operation of compressing a part of the body (e.g., a blood vessel) near the measurement site with a hand or using a separate occlusion device such as a blood pressure cuff.

According to various embodiments, after outputting the guide information, in operation 450, the electronic device 200 (and/or the processor 230) obtains the occluded PPG signal to measure biometric information (e.g., oxygen saturation). The occluded PPG signal may be a PPG signal obtained during and/or after the occlusion operation is performed. According to an embodiment, the blood flow in the body may rapidly decrease due to the occlusion operation, and a DC component having an amount of change increased in inverse proportion to it may be obtained through the occluded PPG signal. For example, in an embodiment, the electronic device 200 may measure biometric information based on a ratio of a difference between a DC component at the time when the occlusion operation starts and a component at the time when the occlusion operation ends.

In this regard, in an embodiment, the electronic device 200 may obtain a part of the obtained PPG signal, for example, a PPG signal obtained after a specified time after outputting the guide information, as an occluded signal. In addition, in an embodiment, the electronic device 200 may limit the operation of the sensor module 210 until a PI requiring an occlusion operation is calculated and guide information is output. In this case, the electronic device 200 may emit the light of the first wavelength and the light of the second wavelength when a specified time elapses after outputting the guide information.

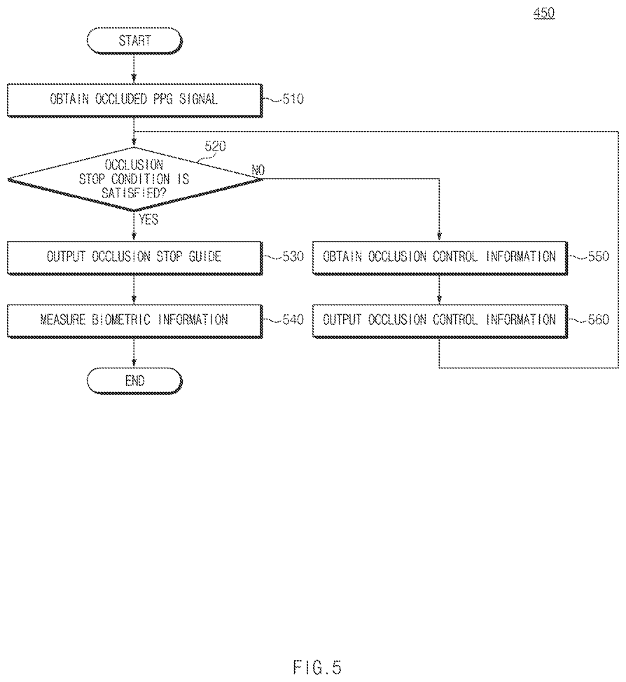

FIG. 5 is a flowchart illustrating an operation of controlling an occlusion operation in an electronic device, according to various embodiments. The operation of FIG. 5 described below may represent various embodiments of the operation 450 of FIG. 4 described above.

Referring to FIG. 5, in operation 510, the electronic device 200 (and/or the processor 230) according to various embodiments may obtain an occluded PPG signal. As described above, the occluded PPG signal may be a PPG signal obtained during and/or after the occlusion operation is performed.

According to an embodiment, the electronic device 200 may output information related to an occlusion operation while obtaining an occluded PPG signal (and/or while performing an occlusion operation). For example, the electronic device 200 may output information notifying that an occlusion operation is performed to obtain biometric information, information notifying that motion is restricted to obtain an occluded PPG signal, and the like.

According to various embodiments, in operation 520, the electronic device 200 (and/or the processor 230) may determine whether the occluded PPG signal satisfies the occlusion stop condition. According to an embodiment, the electronic device 200 may determine whether the variation amount of the DC component of the occluded PPG signal satisfies the occlusion stop condition. For example, in an embodiment, the electronic device 200 may determine that the occlusion stop condition is satisfied in response to detecting the variation amount of a DC component exceeding a reference range (e.g., reference range R of FIG. 2D).

According to various embodiments, when an occlusion stop condition is determined, in operation 530, the electronic device 200 (and/or the processor 230) may output occlusion stop guide (e.g., reference numeral 370 of FIG. 3B). For example, in an embodiment, the electronic device 200 may output at least one of visual guide information, auditory guide information, or tactile guide information indicating occlusion stop. Accordingly, the user may stop the occlusion operation of compressing a part of the body (e.g., blood vessel) near the measurement site with a hand or by using a separate occlusion device such as a blood pressure cuff.

According to various embodiments, after outputting the occlusion stop guide, in operation 540, the electronic device 200 (and/or the processor 230) may measure biometric information based on the occluded PPG signal.

According to various embodiments, when the occlusion stop condition is not determined, the electronic device 200 (and/or the processor 230) may obtain occlusion control information in operation 550. The occlusion control information may relate to occlusion duration and/or occlusion strength. According to an embodiment, the electronic device 200 may obtain information related to occlusion strength (e.g., pressure strength) to increase (or decrease) and/or the occlusion period (e.g., occlusion time) to increase (or decrease) based on the variation amount (or variation rate) of the DC component of the occluded PPG signal. For example, in an embodiment, the electronic device 200 may determine the baseline of the DC component through the occluded PPG signal, and obtain occlusion control information increasing (or decreasing) the occlusion strength and/or the occlusion period when the variation rate of the baseline is equal to or less than (or more than) a specified level.

According to various embodiments, after obtaining occlusion control information, the electronic device 200 (and/or the processor 230) may output the occlusion control information in operation 560. For example, in an embodiment, the electronic device 200 may output at least one of visual occlusion control information, auditory occlusion control information, and tactile occlusion control information. Accordingly, the user may perform an occlusion operation corresponding to the occlusion control information.

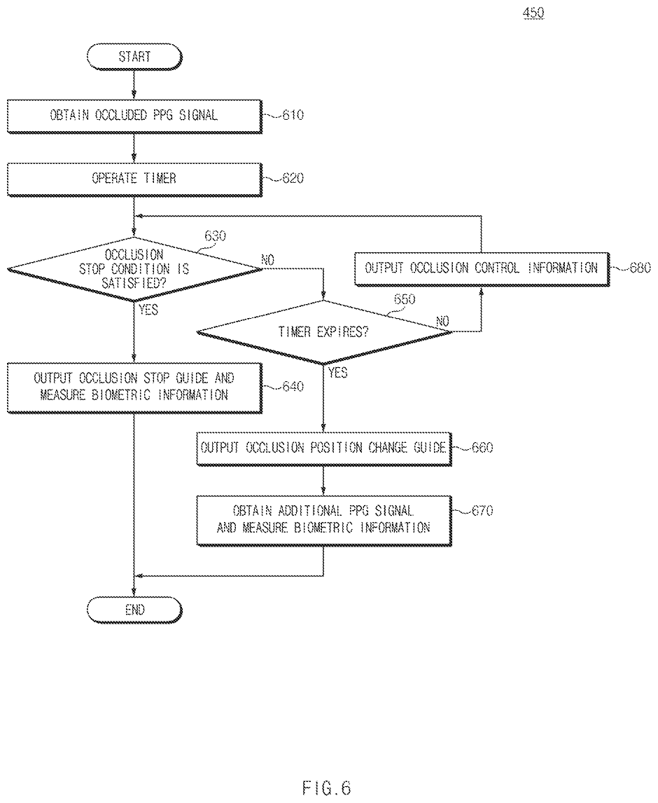

FIG. 6 is a flowchart illustrating another operation of controlling an occlusion operation in an electronic device, according to various embodiments. The operation of FIG. 6 described below may represent various embodiments of at least one of operation 450 of FIG. 4 described above.

Referring to FIG. 6, the electronic device 200 (and/or the processor 230) according to various embodiments may obtain an occluded PPG signal in operation 610. As described above, the occluded PPG signal may be a PPG signal obtained while and/or after the occlusion operation is performed.

According to an embodiment, the electronic device 200 may output information related to an occlusion operation while obtaining an occluded PPG signal (or while performing an occlusion operation). For example, in an embodiment, the electronic device 200 may output information notifying that an occlusion operation is performed to obtain biometric information, information notifying that motion is restricted to obtain an occluded PPG signal, and the like.

According to various embodiments, in operation 620, the electronic device 200 (and/or the processor 230) may operate a timer. The timer may be used to count the specified time to monitor the variation amount of the DC component included in the occluded PPG signal. The running time of this timer may be adjusted by the user.

According to various embodiments, in operation 630, the electronic device 200 (and/or the processor 230) may determine whether the occluded PPG signal satisfies an occlusion stop condition. According to an embodiment, as described above with reference to operation 520 of FIG. 5, the electronic device 200 may determine whether the variation amount of the DC component of the occluded PPG signal satisfies the occlusion stop condition.

According to various embodiments, when the occlusion stop condition is determined, in operation 640, the electronic device 200 (and/or the processor 230) may output an occlusion stop guide and measure biometric information. For example, as described above with reference to operations 530 and 540 of FIG. 5, the electronic device 200 may output guide information instructing occlusion stop and then measure biometric information based on the occluded PPG signal.

According to various embodiments, when the occlusion stop condition is not determined, in operation 650, the electronic device 200 (and/or the processor 230) may determine whether the operation of the timer expires.

According to various embodiments, when the timer operation does not expire, the electronic device 200 (and/or the processor 230) may output the occlusion control information in operation 680. According to an embodiment, as described above with reference to operations 550 and 560 of FIG. 5, the electronic device 200 may obtain and output the occlusion control information.

According to various embodiments, when the operation of the timer expires, in operation 660, the electronic device 200 (and/or the processor 230) may output an occlusion position change guide (e.g., reference numeral 380 in FIG. 3B). According to an embodiment, when the DC component of the PPG signal occluded during a specified time period for which the timer operates does not reach a reference range (e.g., the reference range R of FIG. 2D), the electronic device 200 may determine that the measurement of the biometric information is not possible, and may output an occlusion position change guide. Accordingly, the user may perform the occlusion operation again by changing the occlusion position.

According to various embodiments, after outputting the occlusion position change guide, in operation 670, the electronic device 200 (and/or the processor 230) may obtain an additional PPG signal and measure biometric information. The additional PPG information may be a PPG signal obtained through the sensor module 210 during and/or after the occlusion operation is performed at a changed position.

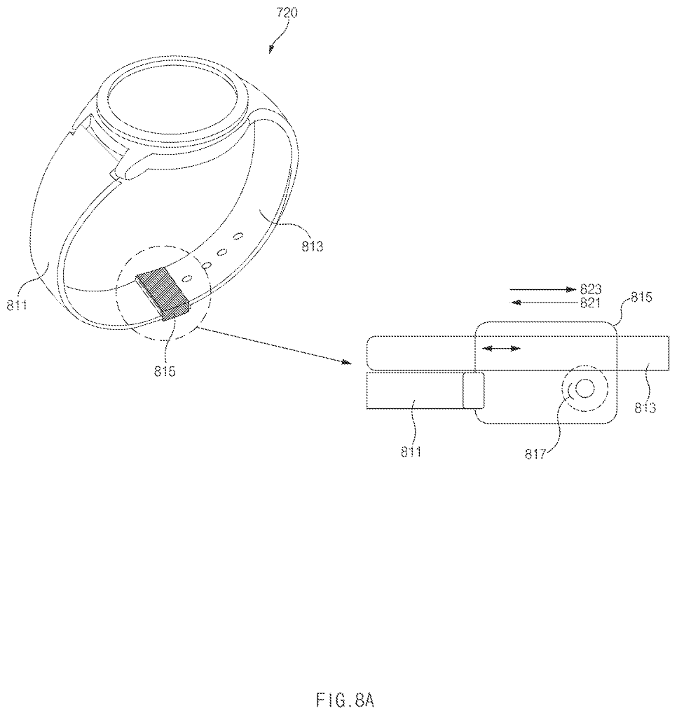

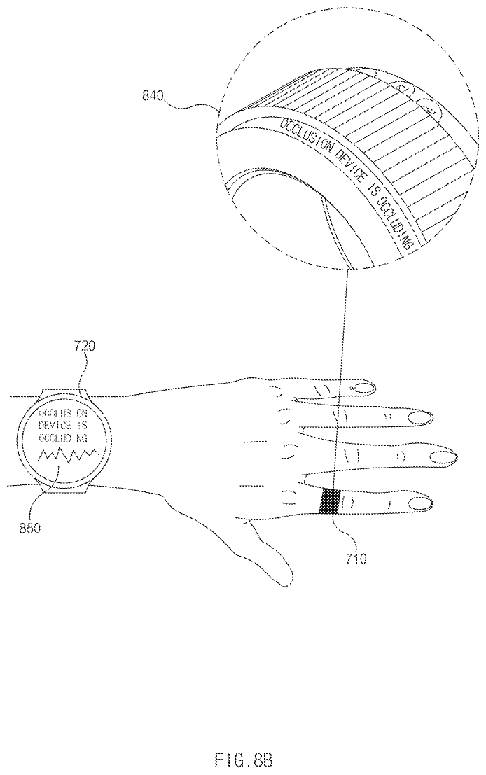

FIG. 7 is a block diagram illustrating a configuration of a biometric information measurement system, according to various embodiments. FIG. 8A is a graphical diagram illustrating an occlusion device, according to various embodiments. FIG. 8B is a graphical diagram illustrating information related to a biometric information obtaining operation provided through an occlusion device, according to various embodiments.

Referring to FIG. 7, a biometric information measurement system, according to various embodiments may include an electronic device 710 and an occlusion device 720 (or an external device). According to an embodiment, as described later with reference to FIG. 8A, the occlusion device 720 may be implemented as a wearable device in the form of a watch, and may provide an occlusion function that compresses a part of the body.

According to an embodiment, the electronic device 710 (e.g., the electronic device 200) may include a sensor module 711 (e.g., the sensor module 210), an output module 713 (e.g., the output module 220), a first processor 715 (e.g., the processor 230), a first communication module 717, and a first memory 719 (e.g., the memory 240). For example, in an embodiment, the sensor module 711, the output module 713, the first processor 717, and the first memory 719 may have similar or identical configurations to those of the electronic device 200 described above with reference to FIG. 2A. Therefore, the details may be omitted.

According to various embodiments, the first communication module 717 may support communication with the occlusion device 720. According to an embodiment, the first communication module 717 may be a device including hardware and software for transmitting and receiving signals (e.g., commands and/or data) between the electronic device 710 and the occlusion device 720.

According to various embodiments, as described above with reference to FIG. 2, the first processor 715 may measure oxygen saturation based on information obtained through the sensor module 711.

According to an embodiment, the first processor 715 may measure oxygen saturation based on the PPG signal detected through the sensor module 711.

According to another embodiment, when an ineffective AC component is detected from the PPG signal, the first processor 715 may measure oxygen saturation based on the occluded PPG signal. As described above, the occluded PPG signal may be a PPG signal obtained through the sensor module 711 during and/or after the occlusion operation is performed.

In this regard, in an embodiment, the first processor 715 may instruct the occlusion device 720 to perform an occlusion operation. According to an embodiment, the first processor 715 may receive information notifying that an occlusion operation is performed from the occlusion device 720 and obtain an occluded PPG signal in response thereto.

According to various embodiments, the occlusion device 720 may include a second communication module 721, a fastening member 723, a driving module 725, a second memory 727, and a second processor 729.

According to various embodiments, the second communication module 721 may be similar to or identical to the first communication module 717 of the electronic device 710. For example, the second communication module 721 may support communication with the electronic device 710.

According to various embodiments, the fastening member 723 may be configured to detach the occlusion device 720 from a part of the user's body (e.g., wrist and/or ankle). The fastening member 723 may include a strap member configured to be bent in a shape surrounding the user's body. For example, as shown in FIG. 8A, in an embodiment, the fastening member may include a first fastening member 811 and a second fastening member 813. In addition, in an embodiment, a containing member 815 may be formed at one end of the first fastening member 811, and the second fastening member 813 may move in a first direction 821 and a second direction 823 within the containing member 815 to adjust the degree of tightness to the body.

According to various embodiments, the driving module 725 may adjust the tightening degree of the fastening member 723 under control of the second processor 729. The driving module 725 may perform an occlusion operation to bring the occlusion device 720 into close contact with the body.

According to an embodiment, as shown in FIG. 8A, the driving module 725 may include an actuator module 817 such as a motor. For example, the driving module 725 may be arranged inside the containing member 815, and may move the second fastening member 813 contained inside the containing member 815 in the first direction 821 or the second direction 823. For example, the driving module 725 may control the actuator module 817 to move the second fastening member 813 in the first direction 821. For example, as the second fastening member 813 moves in the first direction 821, the occlusion device 720 may be worn tightly on the body. As another example, the driving module 725 may control the actuator module 817 to move the second fastening member 813 in the second direction 823. For example, as the second fastening member 813 moves in the second direction 823, the occlusion device 720 may be worn loosely on the body.

According to various embodiments, the second processor 729 may be operatively connected to the second communication module 721, the driving module 725 and the second memory 727, and may control various components (e.g. hardware and/or software components) of the occlusion device 720.

According to an embodiment, the second processor 729 may perform an occlusion operation in response to the occlusion request of the electronic device 710. As described above, in an embodiment, the occlusion operation may be an action of compressing a part of the body. For example, the second processor 729 may control the driving module 725 to perform an occlusion operation of compressing a part of the body by the first fastening member 811 and the second fastening member 813. For example, the second processor 729 may perform an occlusion operation of moving the second fastening member 813 in the first direction 821 such that blood flow in the body is reduced for a specified time period. Similarly, the second processor 729 may perform an occlusion release operation of moving the second fastening member 813 in the second direction 823.

According to various embodiments, the second memory 727 may store commands and/or data related to at least one other element of the occlusion device 720. According to an embodiment, the second memory 727 may include programs, algorithms, routines, and/or instructions related to the biometric information measurement.

According to various embodiments, while the occlusion operation is performed, the electronic device 710 and/or the measurement device 720 may output notification information notifying that the occlusion operation is performed. For example, as shown in FIG. 8B, in an embodiment, the electronic device 710 may output 840 visual notification information notifying that an occlusion operation is performed by the occlusion device 720. Additionally or alternatively, in an embodiment, the occlusion device 720 may also output 850 visual notification information notifying that an occlusion operation is performed by the occlusion device 720. However, this is only illustrative, and embodiments are not limited thereto. For example, in an embodiment, the electronic device 710 and/or the occlusion device 720 may output auditory notification information or tactile notification information.

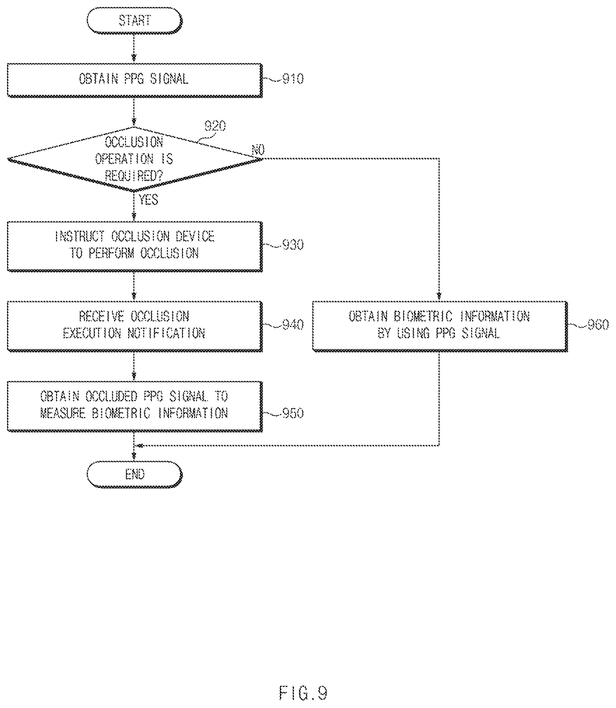

FIG. 9 is a flowchart illustrating another operation of measuring biometric information in an electronic device, according to various embodiments. Each operation in the following embodiment may be performed sequentially, but is not necessarily performed sequentially. For example, the order of each operation may be changed, or at least two operations may be performed in parallel. In addition, at least one of the above operations may be omitted according to an embodiment.

Referring to FIG. 9, the electronic device 710 (and/or the first processor 715) according to various embodiments may obtain a PPG signal in operation 910. According to an embodiment, the electronic device 710 may include the electronic device 101 of FIG. 1.

According to an embodiment, the electronic device 710 may obtain a PPG signal while being worn on a part of the user's body. In this regard, the electronic device 710 may emit light of a first wavelength (e.g., red (wavelength: about 600 nm to about 700 nm)) and light of a second wavelength (e.g., infrared (wavelength: about 780 nm to about 1000 μm)), and may sense the light of the first wavelength and the light of the second wavelength reflected from and/or transmitted through the body.

According to various embodiments, in operation 920, the electronic device 710 (and/or the processor 715) may determine whether an occlusion operation is required. According to an embodiment, as described above, the electronic device 710 may use a perfusion index (PI) calculated based on the PPG signal to determine whether an occlusion operation is necessary.

According to various embodiments, when it is determined that the occlusion operation is unnecessary, the electronic device 710 (and/or the processor 715) may obtain biometric information by using the PPG signal in operation 960. According to an embodiment, the electronic device 710 may measure oxygen saturation based on a component ratio of infrared light and red light extracted from the PPG signal.

According to various embodiments, when it is determined that the occlusion operation is necessary, in operation 930, the electronic device 710 (and/or the processor 715) may instruct the occlusion device 720 to perform the occlusion operation in operation 930.

According to various embodiments, in operation 940, the electronic device 710 (and/or the processor 715) may receive an occlusion execution notification from the occlusion device 720 in response to the occlusion instruction. The occlusion execution notification may be information notifying that an occlusion operation is being performed by the occlusion device 720.

According to various embodiments, the electronic device 710 (and/or the processor 715) may obtain the occluded PPG signal and/or measure biometric information (e.g., oxygen saturation) in operation 950. The occluded PPG signal may be a PPG signal obtained during and/or after the occlusion operation is performed by the occlusion device 720. According to an embodiment, the electronic device 710 may measure biometric information based on a ratio of a difference between a DC component at the time when the occlusion operation starts and a component at the time when the occlusion operation ends.

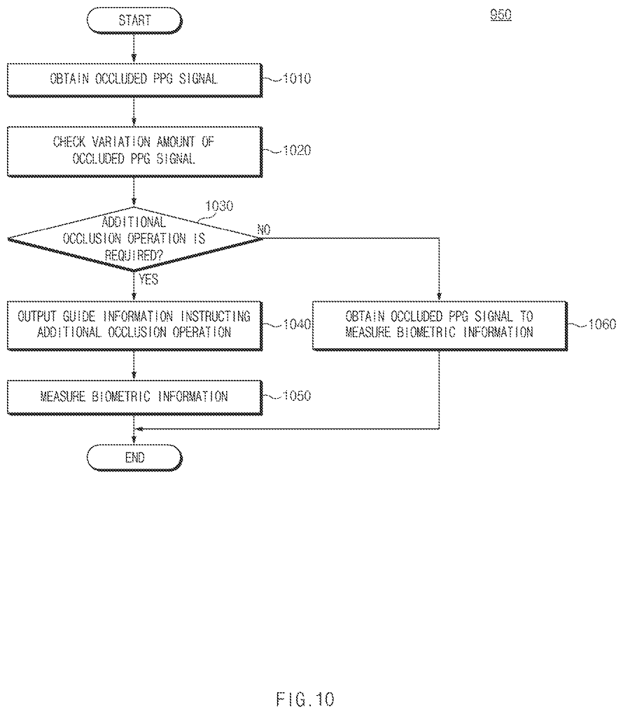

FIG. 10 is a flowchart illustrating an operation of controlling an occlusion operation in an electronic device, according to various embodiments. The operations of FIG. 10 described below may represent various embodiments of the operation 950 of FIG. 9 described above.

Referring to FIG. 10, the electronic device 710 (or the processor 715) according to various embodiments may obtain an occluded PPG signal in operation 1010. As described above, the occluded PPG signal may be a PPG signal obtained during and/or after the occlusion operation is performed by the occlusion device 720.

According to an embodiment, the electronic device 710 may output information related to an occlusion operation while obtaining an occluded PPG signal (or while performing an occlusion operation). For example, in an embodiment, the electronic device 710 may output information notifying that the occlusion operation is being performed to obtain biometric information, information notifying that motion is restricted to obtain the occluded PPG signal, and the like. Accordingly, the user may recognize a situation in which the movement is to be minimized because the occlusion operation is currently performed.

According to various embodiments, in operation 1020, the electronic device 710 (and/or the processor 715) may check the variation amount of the DC component of the occluded PPG signal.

According to various embodiments, in operation 1030, the electronic device 710 (and/or the processor 715) may determine whether an additional occlusion operation is required based on the variation amount of the DC component. The additional occlusion operation may include performing an occlusion operation by using a user and/or a separate additional occlusion device while the occlusion operation is performed by the occlusion device 720.

According to an embodiment, the electronic device 710 may determine that an additional occlusion operation is required when the variation amount of the DC component of the PPG signal that is occluded for a specified time period does not reach a reference range (e.g., the reference range R of FIG. 2D).

According to various embodiments, when it is determined that an additional occlusion operation is not required, in operation 1060, the electronic device 710 (and/or the processor 715) may measure biometric information based on the occluded PPG signal.

According to various embodiments, when it is determined that an additional occlusion operation is required, the electronic device 710 (and/or the processor 715) may output guide information instructing the additional occlusion operation in operation 1040. For example, in an embodiment, the electronic device 710 may output at least one of visual guide information, auditory guide information, and tactile guide information instructing the additional occlusion operation. However, this is only illustrative, and embodiments are not limited thereto. For example, in an embodiment, the electronic device 710 may instruct the occlusion position change before instructing the additional occlusion operation or replacing the additional occlusion operation.

In this regard, in an embodiment, the electronic device 710 may obtain information related to the occlusion device 720 to determine a position where an additional occlusion operation is to be performed. Device information related to the occlusion device 720 may include a kind (type) of the occlusion device 720, a wearing position of the occlusion device 720, and the like.

For example, in an embodiment, when the electronic device 710 determines that the occlusion device 720 is a watch or a band-type wearable device, the electronic device 710 may determine a wrist that does not overlap with the wearing position (e.g., wrist) of the occlusion device 720 as an additional occlusion position and output it together with guide information instructing an additional occlusion operation. In addition, in an embodiment, the electronic device 710 may recognize the arm on which the occlusion device 720 is worn, and may determine a part of the arm on which the occlusion device is worn as an occlusion position in order to increase the occlusion effect.

According to various embodiments, after outputting guide information instructing an additional occlusion operation, in operation 1050, the electronic device 710 (and/or the processor 715) may obtain a PPG signal while the additional occlusion operation is performed and/or may measure biometric information by using it.

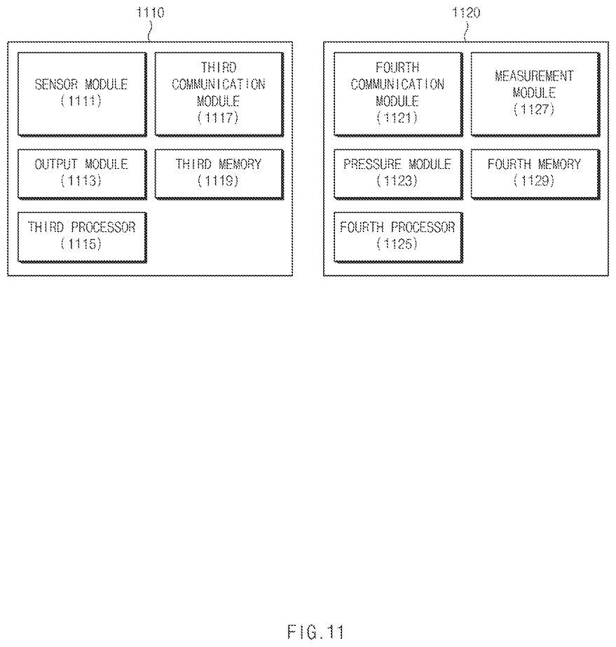

FIG. 11 is a block diagram illustrating another configuration of a biometric information measurement system, according to various embodiments.

Referring to FIG. 11, a biometric information measurement system, according to various embodiments may include an electronic device 1110 and a measurement device 1120. According to an embodiment, the measurement device 1120 may provide an occlusion function for compressing a part of the body and measurement of biometric information. For example, the measurement device 1120 may be a device configured to measure a different type of biometric information from the electronic device 1110 and may be configured to measure oxygen saturation. For example, the measurement device 1120 may include a cuff-type blood pressure monitor configured to measure biometric information while compressing a part of the body by using an occlusion device such as a blood pressure cuff. However, this is only illustrative, and embodiments are not limited thereto. For example, the measurement device 1120 may be a device for measuring the same type of biometric information as the electronic device 1110.

According to an embodiment, the electronic device 1110 (e.g., the electronic device 200) may include a sensor module 1111 (e.g., the sensor module 210), an output module 1113 (e.g., the output module 220), a third processor 1115 (e.g., the processor 230), a third communication module 1117, and a third memory 1119 (e.g., the memory 240). For example, in an embodiment, the sensor module 1111, the output module 1113, the third processor 1117, and the third memory 1119 may be similar to or identical to the configuration of the electronic device 200 described above with reference to FIGS. 2A and 7, and thus the details may be omitted.

According to various embodiments, as described above with reference to FIG. 2, the third processor 1115 may measure oxygen saturation based on information obtained through the sensor module 1111.

According to an embodiment, the third processor 1115 may measure oxygen saturation based on the PPG signal detected through the sensor module 1111.

According to another embodiment, when an ineffective AC component is detected from the PPG signal, the third processor 1115 may measure oxygen saturation based on the occluded PPG signal. As described above, the occluded PPG signal may be a PPG signal obtained through the sensor module 1111 during and/or after the occlusion operation is performed.

In this regard, in an embodiment, the third processor 1115 may receive measurement notification information indicating that biometric information measurement (and/or occlusion operation) starts from the measurement device 1120, and may obtain an occluded PPG signal in response thereto.

According to various embodiments, the measurement device 1120 may include a fourth communication module 1121, a pressure module 1123, a fourth processor 1125, a measurement module 1127, and a fourth memory 1129.

According to various embodiments, the pressure module 1123 may compress a part of the body. According to an embodiment, the pressure module 1123 may be a cuff for measuring blood pressure that touches the subject's upper arm and obtains a signal component by pulse pressure during compression and decompression processes, like a cuff used in a general non-invasive blood pressure measurement system.

According to various embodiments, the measurement module 1127 may measure biometric information, for example, blood pressure, in a state in which a part of the body is pressed by the pressure module 1123.

According to an embodiment, the measurement module 1127 may measure blood pressure in a non-invasive manner, such as a Korotkoff sound, an oscillometric scheme, or the like. However, this is only illustrative, and embodiments are not limited thereto.

According to various embodiments, the fourth processor 1125 may be operatively connected to the fourth communication module 1121, the pressure module 1123, the measurement module 1127, and the fourth memory 1129, and control various components (e.g., hardware and/or software components) of the measurement device 1120.

According to an embodiment, the fourth processor 1125 may transmit measurement notification information to the electronic device 1110 when a biometric information measurement event occurs. As described above, the measurement notification information may be information indicating that biometric information measurement (and/or occlusion operation) is started by the measurement device 1120.

According to an embodiment, the fourth processor 1125 may process to measure biometric information after transmitting measurement notification information.

According to various embodiments, the fourth memory 1129 may store commands and/or data related to at least one other component of the measurement device 200. According to an embodiment, the fourth memory 1129 may include programs, algorithms, routines, and/or instructions related to the biometric information measurement.

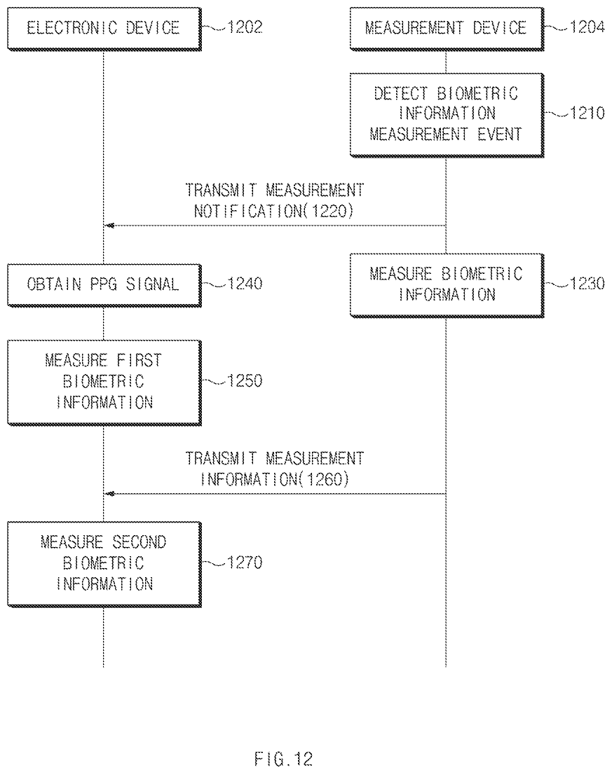

FIG. 12 is a block diagram illustrating a method of measuring biometric information of a biometric information measurement system, according to various embodiments. In the following embodiment, each operation may be performed sequentially, but is not necessarily performed sequentially. For example, the order of each operation may be changed, and at least two operations may be performed in parallel. In addition, at least one of the above-described operations may be omitted according to the embodiment.

Referring to FIG. 12, a biometric information measurement system, according to various embodiments may include an electronic device 1202 (e.g., the electronic device 1110) and a measurement device 1204 (e.g., the measurement device 1120).

According to various embodiments, in operation 1210, the measurement device 1204 may detect a biometric information measurement event. According to an embodiment, the measurement device 1204 may detect a user's input instructing biometric information measurement.

According to various embodiments, in operations 1220 and 1230, the measurement device 1204 may measure biometric information after transmitting a measurement notification to the electronic device 1202. According to an embodiment, the measurement device 1204 may be a blood pressure measurement device that provides an occlusion function. Accordingly, the measurement device 1204 may perform an occlusion operation for measuring biometric information.

According to various embodiments, in operations 1240 and 1250, the electronic device 1202 may obtain a PPG signal and measure first biometric information. According to an embodiment, the first biometric information may include oxygen saturation. For example, in an embodiment, the electronic device 1202 may obtain the PPG signal while the measurement device 1204 performs the occlusion operation and use the PPG signal for the first biometric information measurement.

According to various embodiments, in operation 1260, the measurement device 1204 may transmit measurement information to the electronic device 1202. The measurement information may be blood pressure information measured by the measurement device 1204.

According to various embodiments, in operation 1270, the electronic device 1202 may measure second biometric information based on the received measurement information. The second biometric information may be blood pressure information measured by the electronic device 1202 based on the PPG signal. According to an embodiment, blood pressure measurement performance of the measurement device 1204 may be superior to blood pressure measurement performance of the electronic device 1202. Accordingly, the electronic device 1202 may perform an operation of compensating blood pressure information measured based on the PPG signal by using measurement information provided from the measurement device 1204.

According to various embodiments, an electronic device (e.g., the electronic device 200) may include an output device (e.g., the output module 220), a biometric sensor (e.g., the sensor module 210) including a light emitting unit (e.g., the light emitting unit 212) configured to emit light to a first part of a subject and a light receiving unit (e.g., the light receiving unit 214) configured to receive light emitted by the light emitting unit, and a processor (e.g., the processor 230) operably connected with the output device and the biometric sensor. According to an embodiment, the processor may be configured to obtain a perfusion index based on a photoplethysmogram (PPG) signal obtained through the biometric sensor, measure oxygen saturation by using the PPG signal when the perfusion index corresponds to a first reference, and output guide information instructing an occlusion operation for a second part different from the first part of the subject through the output device when the perfusion index corresponds to a second reference that is lower than the first reference.

According to various embodiments, the processor may be configured to measure the oxygen saturation by using an occluded PPG signal obtained through the biometric sensor after outputting the guide information.

According to various embodiments, the occluded PPG signal may include a PPG signal obtained through the biometric sensor while the occlusion operation corresponding to the guide information is performed.

According to various embodiments, the processor may be configured to determine occlusion control information including at least one of an occlusion period, an occlusion strength, and an occlusion position based on the occluded PPG signal, and output the occlusion control information through the output device.

According to various embodiments, the processor may be configured to obtain an occluded PPG signal obtained through the biometric sensor after outputting the guide information, and output information instructing an occlusion stop through the output device based on the occluded PPG signal.

According to various embodiments, the processor may be configured to instruct the occlusion stop when a variation amount of a DC component of the occluded PPG signal exceeds a specified range.

According to various embodiments, the processor may be configured to obtain an occluded PPG signal obtained through the biometric sensor after outputting the guide information, and output information instructing a change of the occlusion position through the output device based on the occluded PPG signal.

According to various embodiments, the processor may be configured to instruct the change of the occlusion position when a variation amount of a DC component of the occluded PPG signal does not fall within a specified range during a specified time period.

According to various embodiments, the output device may be configured to output at least one piece of visual information, auditory information, and tactile information.

According to various embodiments, a method of operating an electronic device (e.g., the electronic device 200) may include obtaining a photoplethysmogram (PPG) signal of a first part of a subject through a biometric sensor (e.g., the sensor module 210) of the electronic device, obtaining a perfusion index based on the PPG signal, measuring an oxygen saturation by using the PPG signal when the perfusion index corresponds to a first reference, and outputting guide information instructing an occlusion operation for a second part different from the first part of the subject through the output device when the perfusion index corresponds to a second reference that is lower than the first reference.

According to various embodiments, the method may further include measuring the oxygen saturation by using an occluded PPG signal obtained through the biometric sensor after outputting the guide information.

According to various embodiments, the occluded PPG signal may include a PPG signal obtained through the biometric sensor while the occlusion operation corresponding to the guide information is being performed.

According to various embodiments, the method may further include determining occlusion control information including at least one of an occlusion period, an occlusion strength, and an occlusion position based on the occluded PPG signal, and outputting the occlusion control information.

According to various embodiments, the method may further include obtaining an occluded PPG signal obtained through the biometric sensor after outputting the guide information, and outputting information instructing an occlusion stop based on the occluded PPG signal.

According to various embodiments, the method may further include instructing the occlusion stop when a variation amount of a DC component of the occluded PPG signal exceeds a specified range.

According to various embodiments, the method may further include obtaining an occluded PPG signal obtained through the biometric sensor after outputting the guide information, and outputting information instructing a change of an occlusion position based on the occluded PPG signal.

According to various embodiments, the method may further include instructing the change of the occlusion position when a variation amount of a DC component of the occluded PPG signal does not fall within a specified range during a specified time period.

According to various embodiments, the guide information may include at least one piece of visual information, auditory information and tactile information.

According to various embodiments, a biometric information measurement system may include an electronic device (e.g., the electronic device 710) including a biometric sensor (e.g., the sensor module 711) configured to obtain a biometric signal by contacting a first part of a body, and an external device (e.g., the occlusion device 720) including a fastening member (e.g., the fastening member 723) configured to be coupled to a second part of the body different from the first part of the body and a driving module (e.g., the driving module 725) configured to control movement of the fastening member.

According to various embodiments, the electronic device may be configured to measure an oxygen saturation by using the biometric signal when a perfusion index of the biometric signal corresponds to a first reference, and instruct an occlusion operation to the external device when the perfusion index corresponds to a second reference that is lower than the first reference.

According to various embodiments, the external device may be configured to move the fastening member to compress the second part of the body through the driving module in response to an instruction from the electronic device.

According to various embodiments, the external device may further include an output device and be configured to output information indicating that the second part of the body is compressed.

Accordingly, the scope of the various embodiments of the invention should be interpreted to include, in addition to the embodiments disclosed herein, all alterations or modifications derived from the technical ideas of the various embodiments of the invention. Moreover, the embodiment or parts of the embodiments may be combined in whole or in part without departing from the scope of the invention.

1. An electronic device comprising:
an output device;
a biometric sensor including a light emitting unit configured to emit light to a first part of a subject and a light receiving unit configured to receive light emitted by the light emitting unit; and
a processor operably connected with the output device and the biometric sensor,
wherein the processor is configured to:
obtain a perfusion index based on a photoplethysmogram (PPG) signal obtained through the biometric sensor;
measure oxygen saturation by using the PPG signal when the perfusion index corresponds to a first reference; and
output guide information instructing an occlusion operation for a second part of a subject that is different from the first part of the subject through the output device when the perfusion index corresponds to a second reference that is lower than the first reference.

2. The electronic device of claim 1, wherein the processor is configured to measure the oxygen saturation by using an occluded PPG signal obtained through the biometric sensor after outputting the guide information.

3. The electronic device of claim 2, wherein the occluded PPG signal includes a PPG signal obtained through the biometric sensor while the occlusion operation corresponding to the guide information is performed.

4. The electronic device of claim 2, wherein the processor is further configured to:
determine occlusion control information including at least one of an occlusion period, an occlusion strength, and an occlusion position based on the occluded PPG signal; and
output the occlusion control information through the output device.

5. The electronic device of claim 1, wherein the processor is configured to:
obtain an occluded PPG signal obtained through the biometric sensor after outputting the guide information; and
output information instructing an occlusion stop through the output device based on the occluded PPG signal.

6. The electronic device of claim 5, wherein the processor is further configured to instruct the occlusion stop when a variation amount of a DC component of the occluded PPG signal exceeds a specified range.

7. The electronic device of claim 1, wherein the processor is configured to:
obtain an occluded PPG signal obtained through the biometric sensor after outputting the guide information; and
output information instructing a change of an occlusion position through the output device based on the occluded PPG signal, wherein the occlusion position is based on the occluded PPG signal.

8. The electronic device of claim 7, wherein the processor is further configured to instruct the change of the occlusion position when a variation amount of a DC component of the occluded PPG signal does not fall within a specified range during a specified time period.

9. The electronic device of claim 1, wherein the output device is configured to output at least one piece of visual information, auditory information and tactile information.

10. A method of operating an electronic device, the method comprising:
obtaining a photoplethysmogram (PPG) signal of a first part of a subject through a biometric sensor;
obtaining a perfusion index based on the PPG signal;
measuring oxygen saturation by using the PPG signal when the perfusion index corresponds to a first reference; and
outputting guide information instructing an occlusion operation for a second part of the subject that is different from the first part of the subject through the output device when the perfusion index corresponds to a second reference that is lower than the first reference.

11. The method of claim 10, further comprising:
measuring the oxygen saturation by using an occluded PPG signal obtained through the biometric sensor after outputting the guide information.

12. The method of claim 11, wherein the occluded PPG signal includes a PPG signal obtained through the biometric sensor while the occlusion operation corresponding to the guide information is performed.

13. The method of claim 11, further comprising:
determining occlusion control information including at least one of an occlusion period, an occlusion strength, and an occlusion position based on the occluded PPG signal; and
outputting the occlusion control information.

14. The method of claim 10, further comprising:
obtaining an occluded PPG signal obtained through the biometric sensor after outputting the guide information; and
outputting information instructing an occlusion stop based on the occluded PPG signal.

15. The method of claim 14, further comprising:
instructing the occlusion stop when a variation amount of a DC component of the occluded PPG signal exceeds a specified range.

16. The method of claim 10, further comprising:
obtaining an occluded PPG signal obtained through the biometric sensor after outputting the guide information; and
outputting information instructing a change of an occlusion position based on the occluded PPG signal.

17. The method of claim 16, further comprising:
instructing the change of the occlusion position when a variation amount of a DC component of the occluded PPG signal does not fall within a specified range during a specified time period.

18. The method of claim 10, wherein the guide information includes at least one piece of visual information, auditory information and tactile information.

19. A biometric information measurement system comprising:
an electronic device including a biometric sensor configured to obtain a biometric signal by contacting a first part of a body; and
an external device including a fastening member configured to be coupled to a second part of the body that is different from the first part of the body, and a driving module configured to control movement of the fastening member,
wherein the electronic device is configured to:
measure oxygen saturation by using the biometric signal when a perfusion index of the biometric signal corresponds to a first reference, and
instruct the external device to conduct an occlusion operation when the perfusion index corresponds to a second reference that is lower than the first reference,
wherein the external device is configured to move the fastening member to compress the second part of the body through the driving module in response to an instruction of the electronic device.

20. The biometric information measurement system of claim 19, wherein the external device further includes an output device, and
wherein the external device is configured to output information indicating that the second part of the body is compressed.
